(19) United States
(12) Patent Application Publication
(10) Pub. No.: US 2018/0103835 A1
(43) Pub. Date: Apr. 19, 2018

(54) SCANNING-ENDOSCOPE IMAGE EVALUATION SYSTEM

(71) Applicant: OLYMPUS CORPORATION, Tokyo (JP)

(72) Inventors: Masashi YAMADA, Tokyo (JP); Atsuyoshi SHIMAMOTO, Tokyo (JP); Mitsuru NAMIKI, Saitama (JP)

(73) Assignee: OLYMPUS CORPORATION, Tokyo (JP)

(21) Appl. No.: 15/845,349

(22) Filed: Dec. 18, 2017

Related U.S. Application Data
(63) Continuation of application No. PCT/JP2016/067986, filed on Jun. 16, 2016.

(30) Foreign Application Priority Data
Jun. 24, 2015 (JP) PCT/JP2015/068195

(51) Int. Cl. A61B 1/00; G02B 26/10; A61B 1/07; G02B 23/24; G02B 23/26

(52) U.S. Cl. CPC A61B 1/00172; G02B 26/101; A61B 1/00057; G02B 23/2423; G02B 23/26; A61B 1/07

(57) ABSTRACT

A scanning-endoscope image evaluation system includes: a scanning endoscope provided with a light detector and a fiber scanner that includes an optical fiber for guiding illumination light coming from a light source and emitting it from its distal end and an actuator that scans the emitted illumination light by vibrating the distal end of the optical fiber; and a chart for evaluating a characteristic of an image acquired by the scanning endoscope, wherein the distal end of the optical fiber and the light detector are disposed so as to face each other with the chart sandwiched therebetween, and forward scattered light that has been emitted from the optical fiber and that has passed through the chart is detected by the light detector.

### CROSS-REFERENCE TO RELATED APPLICATIONS

This application is a Continuation Application of International Application No. PCT/2016/067986 filed on Jun. 16, 2016, which claims priority to International Application No. PCT/JP2015/068195 filed on Jun. 24, 2015. The contents of International Application No. PCT/2016/067986 and International Application No. PCT/JP2015/068195 are hereby incorporated herein by reference in their entirety.

### TECHNICAL FIELD

The present invention relates to a scanning-endoscope image evaluation system.

### BACKGROUND ART

There is a known scanning endoscope in which an emitting end of an optical fiber that guides light coming from a light source is vibrated to two-dimensionally scan the light emitted from the emitting end on an imaging subject, and an image is acquired by receiving light returning from individual scanning positions on the imaging subject (for example, see PTL 1).

With this scanning endoscope, a light-receiving optical fiber that points in the same direction as the emitting end is secured at the radial outside of the emitting end of the optical fiber that emits the light, and thus, the light returning from the imaging subject toward the emitting end is received and collected thereby.

### CITATION LIST

### Patent Literature

{PTL 1} Publication of Japanese Patent No. 5608718

### SUMMARY OF INVENTION

An aspect of the present invention is a scanning-endoscope image evaluation system including: a scanning endoscope provided with a light detector and a fiber scanner that includes an optical fiber for guiding illumination light coming from a light source and emitting the illumination light from its distal end and an actuator which makes the emitted illumination light scan by vibrating the distal end of the optical fiber; and a chart provided with an index for evaluating a characteristic of an image acquired by the scanning endoscope, wherein the distal end of the optical fiber and the light detector are disposed so as to face each other with the chart sandwiched therebetween, and forward scattered light that has been emitted from the distal end of the optical fiber and that has passed through the chart is detected by the light detector.

### BRIEF DESCRIPTION OF DRAWINGS

FIG. 1 is an overall configuration diagram showing a scanning-endoscope image evaluation system according to an embodiment of the present invention.

FIG. 2 is an overall configuration diagram showing a scanning endoscope in FIG. 1.

FIG. 3 is a perspective view showing a fiber scanner of the scanning-endoscope image evaluation system in FIG. 1.

FIG. 4 is a diagram showing an example of the pattern of illumination light manipulated via the fiber scanner of the scanning-endoscope image evaluation system in FIG. 1.

FIG. 5 is a partial longitudinal sectional view showing an example of a chart provided in the scanning-endoscope image evaluation system in FIG. 1.

FIG. 6 is a partial longitudinal sectional view showing another example of the chart provided in the scanning-endoscope image evaluation system in FIG. 1.

### DESCRIPTION OF EMBODIMENT

An image evaluation system 1 of a scanning endoscope 20 according to an embodiment of the present invention will be described below with reference to the drawings.

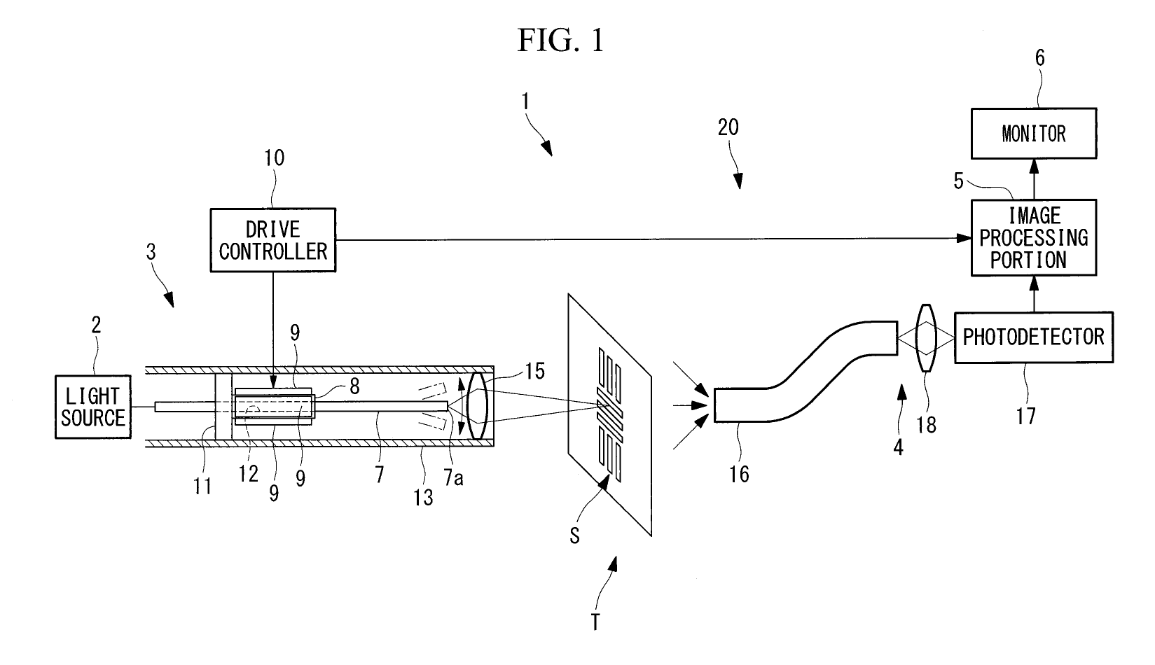

As shown in FIG. 1, the image evaluation system 1 of the scanning endoscope 20 according to this embodiment is provided with the scanning endoscope 20 that acquires an image by scanning illumination light (for example, visible light, infrared light, excitation light, or the like) coming from a light source 2, and a transmissive chart (imaging subject) T that has an index pattern (index) S for evaluating the characteristics of the image acquired by the scanning endoscope 20.

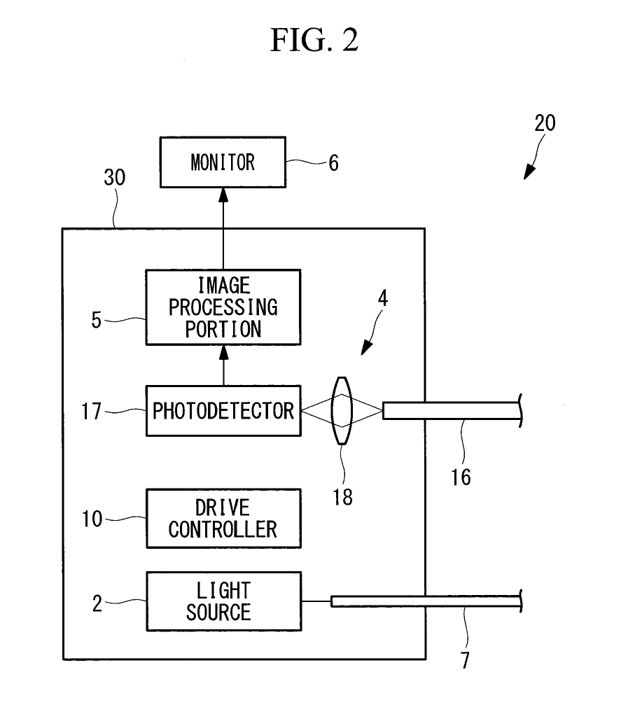

In addition, the scanning endoscope 20 according to this embodiment is provided with: the light source 2 that generates the illumination light; a fiber scanner 3 that scans the illumination light coming from the light source 2; a light detector 4 that detects light (for example, forward scattered light, fluorescence, or direct light); an image-processing portion 5 that generates an image of the chart T on the basis of the intensity of the light detected by the light detector 4; and a monitor 6 that displays the generated image. In addition, as shown in FIG. 2, the light source portion 2, the light detector 4, the image-processing portion 5, and a drive controller 10, described later, are provided in a housing 30 of the scanning endoscope 20.

The fiber scanner 3 is provided with: an optical fiber 7 that guides the illumination light coming from the light source 2 and emits the light from a distal end 7a thereof; a tubular vibration transmitting member 8 that supports, in a state in which the optical fiber 7 is made to pass therethrough, the optical fiber 7 at a position distant from the distal end 7a of the optical fiber 7 by a predetermined distance; four piezoelectric elements (actuators) 9 that are attached to outer surfaces of the vibration transmitting member 8 at equal intervals in the circumferential direction; and the drive controller 10 that adjusts AC voltages applied to the piezoelectric elements 9. In addition, the fiber scanner 3 is provided with, at a rear end of the vibration transmitting member 8, a circular holder portion 11 that secures the vibration transmitting member 8 and the optical fiber 7 that is made to pass through the vibration transmitting member 8.

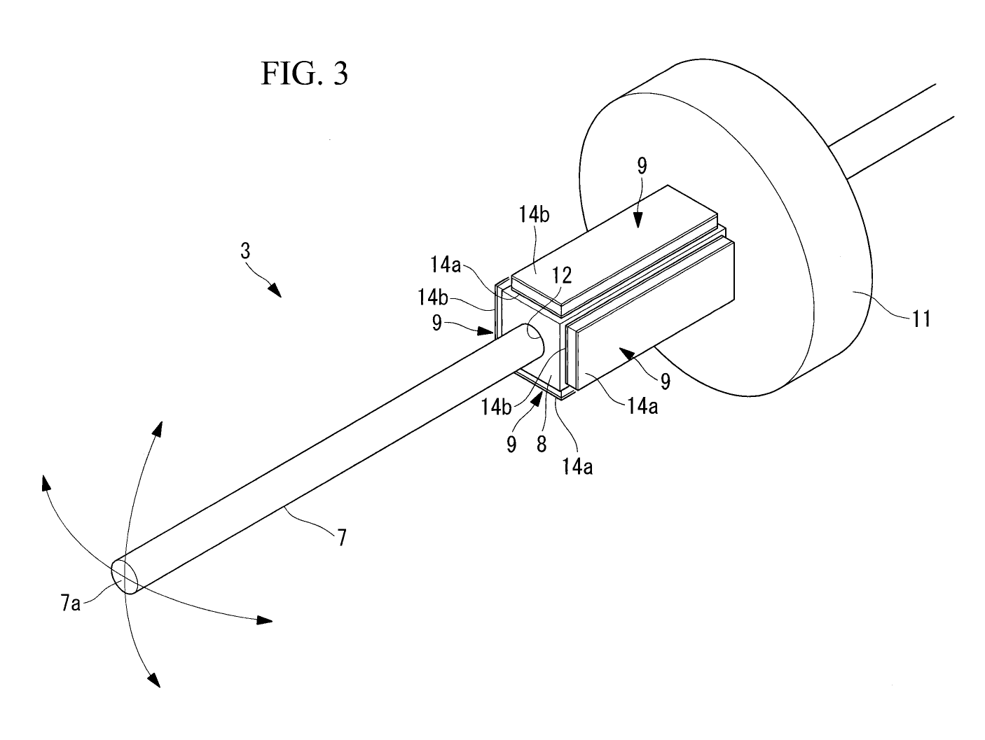

The vibration transmitting member 8 is provided with a conductive metal material at least on the surfaces thereof, and has, as shown in FIG. 3, a shape in which a through-hole 12 through which the optical fiber 7 can be made to pass is formed along a longitudinal axis of a right rectangular column thereof.

The holder portion 11 is secured to an outer-cylinder member 13.

The piezoelectric elements 9 are formed in a flat plate shape in which electrodes 14a and 14b are provided at two end surfaces in the thickness direction thereof, and the electrodes 14a are secured to the respective side surfaces of the right-rectangular-column portion of the vibration transmitting member 8 in a state in which the electrodes 14a are in electrical contact therewith. The two pairs of piezoelectric elements 9 that are disposed at opposing positions with the optical fiber 7 sandwiched therebetween are disposed so that the polarization directions thereof are oriented in the same direction. AC voltages of the same phase are supplied to the piezoelectric elements 9 that are disposed at the opposing positions with the optical fiber 7 sandwiched therebetween.

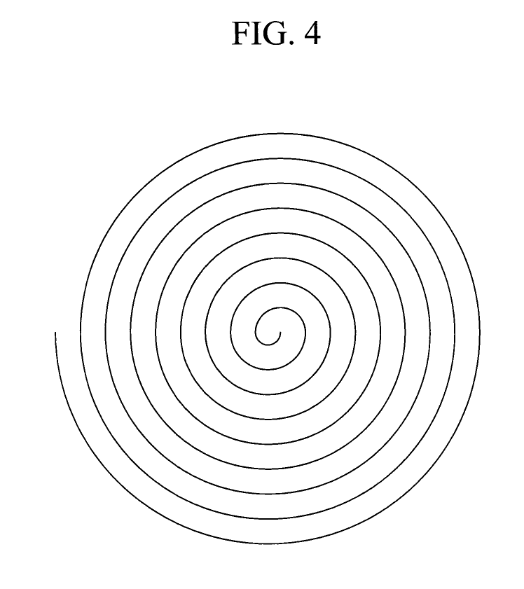

The drive controller 10 is configured to apply, to the two pairs of piezoelectric elements 9, the AC voltages, which oscillate at a constant frequency, by making the phases thereof differ by 90° while changing the amplitudes thereof in a sine-wave manner. In other words, by applying the AC voltages to the respective pairs of piezoelectric elements 9, the optical fiber 7 is made to undergo bending vibrations by means of stretching vibrations of the respective pairs of piezoelectric elements 9, and, by doing so, the distal end 7a of the optical fiber 7 is displaced in a spiral manner, as shown in FIG. 4, thus scanning the illumination light emitted from the distal end 7a of the optical fiber 7 in a spiral manner.

In FIG. 1, reference sign 15 is a focusing lens. The optical fiber 7 is, for example, a single-mode fiber.

In addition, the drive controller 10 transmits information that indicates scanning positions of the illumination light to the image-processing portion 5.

The light detector 4 is formed as a component separated from the fiber scanner 3 and is provided with one or more light-receiving optical fibers (optical fibers) 16 that receive, at distal ends thereof, light generated at the chart T, and a photodetector(s) 17, such as a photomultiplier tube or the like, that detects the light received by the light-receiving optical fiber(s) 16. In the figure, reference sign 18 is a focusing lens that focuses the light detected by the light-receiving optical fiber 16 on the photodetector 17.

In this embodiment, the light-receiving optical fiber 16 of the light detector 4 is disposed on the opposite side of the fiber scanner 3 so as to sandwich the chart T therebetween. The light-receiving optical fiber 16 is, for example, a multi-mode fiber. Two or more optical fibers may be bundled or a fiber bundle may be employed so as to serve as the light-receiving optical fiber 16.

The image-processing portion 5 generates an image by associating the individual positions scanned by the fiber scanner 3 by using the illumination light and the intensity of light detected by the photodetector 17 when the illumination light is radiated onto the individual scanning positions. The generated image is displayed on the monitor 6.

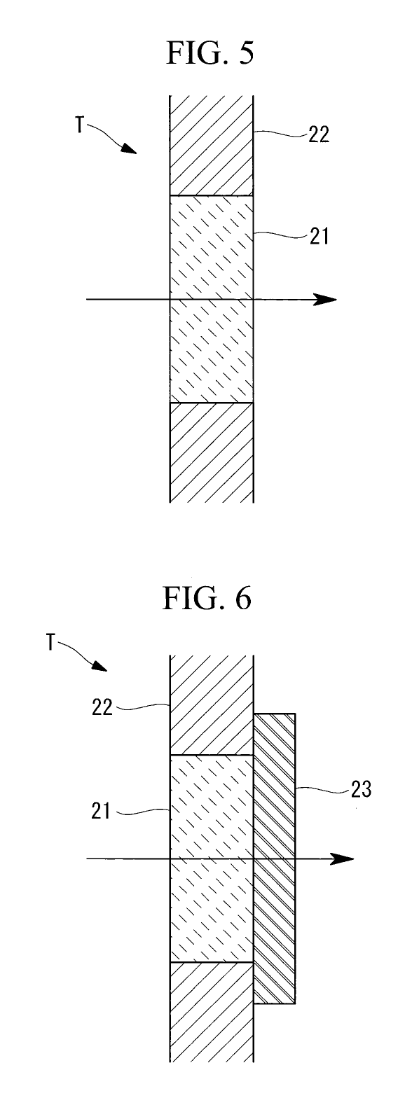

As shown in FIG. 5, a chart in which an index pattern S is formed by using a transparent material (transmitting member) 21 that allows the illumination light to pass therethrough and a light shielding material 22 that blocks the illumination light may be used as the transmissive chart T. Note that the arrow in the figure indicates the direction in which the illumination light passes through. Also, one of the index pattern S and the portion other than the index pattern S may be formed of the transparent material 21, and the other may be formed of the light shielding material 22.

In addition, in order to prevent the illumination light that has passed through the index pattern S formed of the transparent material 21 from being directly received, the transparent material 21 may be a scatterer (light scatterer, for example, paper, a fluorescent substance, or the like) such as a filter or the like. In addition, as shown in FIG. 6, a scatterer 23 may be disposed on a surface of the chart T on the light-receiving optical fiber 16 side.

For example, in the case in which the illumination light that has passed through the index pattern S is directly received, because the light level detected by the light detector 4 is decreased by providing a light-level cut filter or the like in the scatterer 23, it is possible to prevent saturation in the image. In addition, by providing a wavelength cut filter in the scatterer 23, it is possible to detect only the light at a desired wavelength in forward scattered light by using the light detector 4. Furthermore, in the case in which excitation light is used as the illumination light, by providing a fluorescent substance in the scatterer 23, it is possible to detect excited fluorescence. Note that these scatterers 23 may be provided as a combination.

Examples of the transmissive chart T include a lattice chart, a dot chart, a viewing-angle chart, and a resolution chart. Because different image characteristics are obtained by using these charts, different charts can be used in accordance with the characteristics to be evaluated. Specifically, image distortion can be evaluated by using a lattice chart and a dot chart, the image viewing angle can be evaluated by using a viewing-angle chart, and image resolution can be evaluated by using a resolution chart.

The operation of the thus-configured image evaluation system 1 of the scanning endoscope 20 according to this embodiment will be described below.

In order to evaluate an image acquired by the scanning endoscope 20 by using the image evaluation system 1 of the scanning endoscope 20 according to this embodiment, the distal end of the fiber scanner 3 is made to face the chart T, as shown in FIG. 1. In addition, the distal end of the light-receiving optical fiber 16 is placed at a position facing the distal end of the fiber scanner 3 with the chart T sandwiched therebetween.

In this state, the illumination light is generated in the light source 2, and the actuators 9 are driven by means of the drive controller 10. By doing so, the illumination light that comes from the light source 2 and that is guided by the optical fiber 7 is emitted toward the chart T from the distal end 7a of the optical fiber 7, and is scanned, for example, in a spiral manner by means of the vibrations of the distal end 7a of the optical fiber 7.

By scanning the illumination light, although light generated at the individual scanning positions in the chart T is scattered in all directions, a portion of forward scattered light that has passed through the chart T is received at the distal end of the light-receiving optical fiber 16 in the light detector 4, and thus, the intensity thereof is detected by the photodetector 17. The light detected by the photodetector 17 is transmitted to the image-processing portion 5. Because the information indicating the scanning positions of the illumination light is transmitted to the image-processing portion 5 from the drive controller 10, an image is generated by storing the information about the intensity of the light detected by the photodetector 17 and the information about the scanning positions in association with each other. The generated image is displayed on the monitor 6.

In this case, with the image evaluation system 1 of the scanning endoscope 20 according to this embodiment, because the fiber scanner 3 that emits the illumination light and the light detector 4 that receives the light are disposed at opposing positions with respect to the chart T sandwiched therebetween, it is possible to reliably prevent the illumination light reflected at a surface of the chart T from being detected by the light detector 4. As compared to the related art in which a system that detects backscattered light is employed and an image is deteriorated due to high-intensity stray light generated by light reflected at a surface of an imaging subject, it is possible to generate an image that precisely represents the chart T by reliably preventing image deterioration. As a result, there is an advantage in that it is possible to evaluate image characteristics in a highly precise manner on the basis of the acquired image. The image characteristics refer to, for example, viewing angle, distortion, resolution, or the like, and, by obtaining displacement or distortion with respect to the index S of the chart T by using the generated image, it is possible to perform device calibration on the basis of that information.

In addition, because the fiber scanner 3 and the light detector 4 are formed as separate components, as compared with the scanning endoscope in the related art in which the two components are integrated, there is an advantage in that it is possible to reduce the diameter of each of the fiber scanner 3 and the light detector 4.

Note that, as the calibration method, the present invention is also effective as a calibration method for the scanning endoscope in the related art employing the system in which backscattered light is detected. In other words, regarding a calibration image, forward scattered light received by the light-receiving optical fiber 16, which is disposed so as to face the fiber scanner 3 with the chart T sandwiched therebetween, may be utilized instead of utilizing backscattered light received by the light-receiving fiber provided in the scanning endoscope.

The inventors have arrived at the following aspects of the present invention.

An aspect of the present invention is a scanning-endoscope image evaluation system including: a scanning endoscope provided with a light detector and a fiber scanner that includes an optical fiber for guiding illumination light coming from a light source and emitting the illumination light from its distal end and an actuator which makes the emitted illumination light scan by vibrating the distal end of the optical fiber; and a chart provided with an index for evaluating a characteristic of an image acquired by the scanning endoscope, wherein the distal end of the optical fiber and the light detector are disposed so as to face each other with the chart sandwiched therebetween, and forward scattered light that has been emitted from the distal end of the optical fiber and that has passed through the chart is detected by the light detector.

With this aspect, the fiber scanner is made to face one side of the chart, the light detector is made to face the other side of the chart, the illumination light coming from the light source is guided by the optical fiber, the distal end of the optical fiber is made to vibrate by driving the actuator, and thus, the illumination light emitted from the distal end of the optical fiber is scanned on the chart. At the chart, although the light generated at the individual positions at which the illumination light is scanned is scattered in all directions the forward scattered light of the illumination light that is scattered in the direction in which light passes through the index of the chart is detected by the light detector that is disposed on the opposite side of the fiber scanner with respect to the chart sandwiched therebetween.

By doing so, by storing information about the intensity of the light detected by the light detector and information about the individual positions scanned by the fiber scanner in association with each other, it is possible to generate an image of the chart.

In this case, because the light detector detects only the forward scattered light that has passed through the index of the chart, it is possible to prevent detection of the reflected light, which is the illumination light that is emitted from the distal end of the optical fiber and that returns after being reflected at a surface of the chart. In other words, because the acquired image does not include the reflected light, which is high-intensity stray light, the image precisely represents the chart, and thus, it is possible to precisely evaluate the characteristics of the acquired image.

In the above-described aspect, one of the index and the rest portion in the chart may be formed of a transmitting member that allows light to pass therethrough, and the other of the index and the rest portion in the chart may be formed of a light shielding member.

In addition, in the above-described aspect, the index may be provided with a light scatterer.

In addition, the light scatterer may be disposed on a surface of the transmitting member.

The aforementioned aspects afford an advantage in that it is possible to precisely evaluate the characteristics of an acquired image without being affected by light reflected at a surface of an imaging subject.

### REFERENCE SIGNS LIST

1 image evaluation system
2 light source
3 fiber scanner
4 light detector
7 optical fiber
7a distal end
9 piezoelectric element (actuator)
16 light-receiving optical fiber (optical fiber)
20 scanning endoscope
21 transparent material (transmitting member)
23 scatterer (light scatterer)
S index pattern (index)
T chart

1. A scanning-endoscope image evaluation system comprising:
a scanning endoscope provided with a light detector and a fiber scanner that includes an optical fiber for guiding illumination light coming from a light source and emitting the illumination light from its distal end and an actuator which makes the emitted illumination light scan by vibrating the distal end of the optical fiber; and
a chart provided with an index for evaluating a characteristic of an image acquired by the scanning endoscope,
wherein the distal end of the optical fiber and the light detector are disposed so as to face each other with the chart sandwiched therebetween, and forward scattered light that has been emitted from the distal end of the optical fiber and that has passed through the chart is detected by the light detector.

2. The scanning-endoscope image evaluation system according to claim 1, wherein one of the index and the rest portion in the chart is formed of a transmitting member that allows light to pass therethrough, and the other of the index and the rest portion in the chart is formed of a light shielding member.

3. The scanning-endoscope image evaluation system according to claim 1, wherein the index is provided with a light scatterer.

4. The scanning-endoscope image evaluation system according to claim 2, wherein the index is provided with a light scatterer.

5. The scanning-endoscope image evaluation system according to claim 3, wherein the light scatterer is disposed on a surface of the transmitting member.

6. The scanning-endoscope image evaluation system according to claim 4, wherein the light scatterer is disposed on a surface of the transmitting member.
